(19) United States
(12) Patent Application Publication
(10) Pub. No.: US 2025/0062003 A1
(43) Pub. Date: Feb. 20, 2025

(54) RECOMMENDING ITEMS OR RECIPES BASED ON HEALTH CONDITIONS ASSOCIATED WITH ONLINE SYSTEM USERS

(71) Applicant: Maplebear Inc., San Francisco, CA (US)

(72) Inventors: Nour Alkhatib, Mississauga (CA); Karuna Ahuja, San Francisco, CA (US)

(21) Appl. No.: 18/234,070

(22) Filed: Aug. 15, 2023

(51) Int. Cl. G16H 20/60; G06F 40/40; G06Q 10/087; G06Q 30/0601

(52) U.S. Cl. CPC G16H 20/60; G06F 40/40; G06Q 10/087; G06Q 30/0631

(57) ABSTRACT

An online system retrieves historical interaction data for a user describing objects with which the user previously interacted and health data associated with the user. The system accesses and applies a multiclass classification model to classify whether the user has each of a set of health conditions based on the historical interaction and health data. The system generates a prompt including a set of classes associated with the user and a request for a set of objects appropriate for the user, in which the set of classes indicates whether the user has each health condition and an appropriateness of an object is based on whether the user has each health condition. The system provides the prompt to a large language model to obtain a textual output, extracts one or more objects (e.g., items and/or recipes) from the output, and sends a recommendation for the object(s) for display to the user.

### BACKGROUND

Online systems, such as online concierge systems, may make various recommendations to their users (e.g., for items, recipes, etc. that the users are most likely to find relevant) based on information that is specific to each user. For example, an online system may identify items and recipes for recommendation to a user based on historical data describing the user's interaction history with the online system (e.g., items included in previous orders placed by the user with the online system and recipes the user previously saved), the user's preferences (e.g., for certain types of cuisines), etc. In this example, the online system may identify items the user is likely to purchase again and recipes the user is likely to try again, select one or more of them (e.g., based on a predicted affinity of the user for each, based on predicted availabilities of each item, etc.), and present them to the user.

However, some recommendations may not be appropriate for online system users due to health conditions the users may have. For example, suppose that a user of an online system who suffers from hypertension, heart disease, and diabetes frequently places orders with the online system including foods that are high in sugar, sodium, and saturated fat, and frequently saves recipes presented to them by the online system that are also high in sugar, sodium, and saturated fat. In this example, based on the user's interaction history with the online system, the online system may recommend more of the foods that the user frequently purchases and recipes that the user frequently saves, which may worsen the user's health conditions. As such, failure to recommend healthier options to online system users may have a negative impact on the users' health.

### SUMMARY

In accordance with one or more aspects of the disclosure, an online system recommends items or recipes to online system users based on health conditions associated with the users. More specifically, an online system retrieves a set of historical interaction data for a user of the online system describing a set of objects with which the user previously interacted and a set of health data associated with the user. The online system then accesses a multiclass classification model trained to classify whether the user has each of a set of health conditions and applies the model. The online system also generates a prompt including a set of classes associated with the user and a request for a set of objects appropriate for the user, in which the set of classes indicates whether the user has each health condition and an appropriateness of an object for the user is based at least in part on whether the user has each health condition. The online system provides the prompt to a large language model to obtain a textual output and extracts, from the textual output, one or more objects for recommendation to the user, in which the object(s) include one or more items and/or recipes. The online system then sends a recommendation for the object(s) for display to a client device associated with the user.

### BRIEF DESCRIPTION OF THE DRAWINGS

FIG. 1 illustrates an example system environment for an online system, in accordance with one or more embodiments.

FIG. 2 illustrates an example system architecture for an online system, in accordance with one or more embodiments.

FIG. 3 is a flowchart of a method for recommending items or recipes based on health conditions associated with online system users, in accordance with one or more embodiments.

FIG. 4 is an interaction diagram of a method for recommending items or recipes based on health conditions associated with online system users, in accordance with one or more embodiments.

FIG. 5 illustrates an example of health trend scores for a user of an online system, in accordance with one or more embodiments.

### DETAILED DESCRIPTION

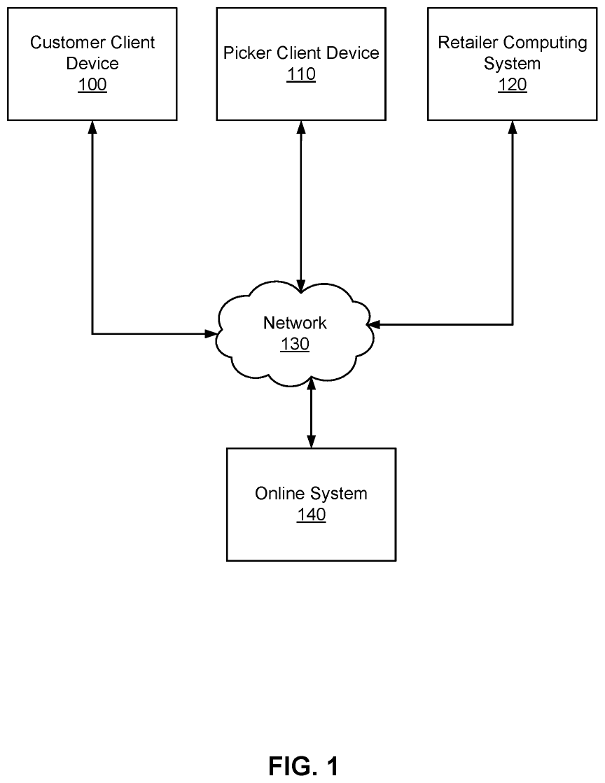

FIG. 1 illustrates an example system environment for an online system 140, such as an online concierge system, in accordance with one or more embodiments. The system environment illustrated in FIG. 1 includes a customer client device 100, a picker client device 110, a retailer computing system 120, a network 130, and an online system 140. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

As used herein, customers, pickers, and retailers may be generically referred to as “users” of the online system 140. Additionally, while one customer client device 100, picker client device 110, and retailer computing system 120 are illustrated in FIG. 1, any number of customers, pickers, and retailers may interact with the online system 140. As such, there may be more than one customer client device 100, picker client device 110, or retailer computing system 120.

The customer client device 100 is a client device through which a customer may interact with the picker client device 110, the retailer computing system 120, or the online system 140. The customer client device 100 can be a personal or mobile computing device, such as a smartphone, a tablet, a laptop computer, or a desktop computer. The customer client device 100 also may be a wearable device (e.g., a smartwatch, a fitness tracker, etc.) or another device (e.g., a smart scale) that may interact with the online system 140 either directly or indirectly (e.g., via a Bluetooth connection with another customer client device 100 or a third-party system, such as a fitness tracking application, that communicates with the online system 140). In some embodiments, the customer client device 100 executes a client application that uses an application programming interface (API) to communicate with the online system 140.

A customer uses the customer client device 100 to place an order with the online system 140. An order specifies a set of items to be delivered to the customer. An “item,” as used herein, refers to a good or product that may be provided to the customer through the online system 140. The order may include item identifiers (e.g., a stock keeping unit or a price look-up code) for items to be delivered to the customer and may include quantities of the items to be delivered. Additionally, an order may further include a delivery location to which the ordered items are to be delivered and a timeframe during which the items should be delivered. In some embodiments, the order also specifies one or more retailers from which the ordered items should be collected.

The customer client device 100 presents an ordering interface to the customer. The ordering interface is a user interface that the customer can use to place an order with the online system 140. The ordering interface may be part of a client application operating on the customer client device 100. The ordering interface allows the customer to search for items that are available through the online system 140 and the customer can select which items to add to a “shopping list.” A “shopping list,” as used herein, is a tentative set of items that the customer has selected for an order but that has not yet been finalized for an order. The ordering interface allows a customer to update the shopping list, e.g., by changing the quantity of items, adding or removing items, or adding instructions for items that specify how the items should be collected.

The customer client device 100 may receive additional content from the online system 140 to present to a customer. For example, the customer client device 100 may receive coupons, recipes, or item suggestions. The customer client device 100 may present the received additional content to the customer as the customer uses the customer client device 100 to place an order (e.g., as part of the ordering interface).

Additionally, the customer client device 100 includes a communication interface that allows the customer to communicate with a picker that is servicing the customer's order. This communication interface allows the customer to input a text-based message to transmit to the picker client device 110 via the network 130. The picker client device 110 receives the message from the customer client device 100 and presents the message to the picker. The picker client device 110 also includes a communication interface that allows the picker to communicate with the customer. The picker client device 110 transmits a message provided by the picker to the customer client device 100 via the network 130. In some embodiments, messages sent between the customer client device 100 and the picker client device 110 are transmitted through the online system 140. In addition to text messages, the communication interfaces of the customer client device 100 and the picker client device 110 may allow the customer and the picker to communicate through audio or video communications, such as a phone call, a voice-over-IP call, or a video call.

The picker client device 110 is a client device through which a picker may interact with the customer client device 100, the retailer computing system 120, or the online system 140. The picker client device 110 can be a personal or mobile computing device, such as a smartphone, a tablet, a laptop computer, or a desktop computer. In some embodiments, the picker client device 110 executes a client application that uses an application programming interface (API) to communicate with the online system 140.

The picker client device 110 receives orders from the online system 140 for the picker to service. A picker services an order by collecting the items listed in the order from a retailer location. The picker client device 110 presents the items that are included in the customer's order to the picker in a collection interface. The collection interface is a user interface that provides information to the picker identifying items to collect for a customer's order and the quantities of the items. In some embodiments, the collection interface provides multiple orders from multiple customers for the picker to service at the same time from the same retailer location. The collection interface further presents instructions that the customer may have included related to the collection of items in the order. Additionally, the collection interface may present a location of each item in the retailer location, and may even specify a sequence in which the picker should collect the items for improved efficiency in collecting items. In some embodiments, the picker client device 110 transmits to the online system 140 or the customer client device 100 which items the picker has collected in real time as the picker collects the items.

The picker can use the picker client device 110 to keep track of the items that the picker has collected to ensure that the picker collects all of the items for an order. The picker client device 110 may include a barcode scanner that can determine an item identifier encoded in a barcode coupled to an item. The picker client device 110 compares this item identifier to items in the order that the picker is servicing, and if the item identifier corresponds to an item in the order, the picker client device 110 identifies the item as collected. In some embodiments, rather than or in addition to using a barcode scanner, the picker client device 110 captures one or more images of the item and determines the item identifier for the item based on the images. The picker client device 110 may determine the item identifier directly or by transmitting the images to the online system 140. Furthermore, the picker client device 110 determines a weight for items that are priced by weight. The picker client device 110 may prompt the picker to manually input the weight of an item or may communicate with a weighing system in the retailer location to receive the weight of an item.

When the picker has collected all of the items for an order, the picker client device 110 provides instructions to a picker for delivering the items for a customer's order. For example, the picker client device 110 displays a delivery location from the order to the picker. The picker client device 110 also provides navigation instructions for the picker to travel from the retailer location to the delivery location. If a picker is servicing more than one order, the picker client device 110 identifies which items should be delivered to which delivery location. The picker client device 110 may provide navigation instructions from the retailer location to each of the delivery locations. The picker client device 110 may receive one or more delivery locations from the online system 140 and may provide the delivery locations to the picker so that the picker can deliver the corresponding one or more orders to those locations. The picker client device 110 may also provide navigation instructions for the picker from the retailer location from which the picker collected the items to the one or more delivery locations.

In some embodiments, the picker client device 110 tracks the location of the picker as the picker delivers orders to delivery locations. The picker client device 110 collects location data and transmits the location data to the online system 140. The online system 140 may transmit the location data to the customer client device 100 for display to the customer such that the customer can keep track of when their order will be delivered. Additionally, the online system 140 may generate updated navigation instructions for the picker based on the picker's location. For example, if the picker takes a wrong turn while traveling to a delivery location, the online system 140 determines the picker's updated location based on location data from the picker client device 110 and generates updated navigation instructions for the picker based on the updated location.

In one or more embodiments, the picker is a single person who collects items for an order from a retailer location and delivers the order to the delivery location for the order. Alternatively, more than one person may serve the role as a picker for an order. For example, multiple people may collect the items at the retailer location for a single order. Similarly, the person who delivers an order to its delivery location may be different from the person or people who collected the items from the retailer location. In these embodiments, each person may have a picker client device 110 that they can use to interact with the online system 140. Additionally, while the description herein may primarily refer to pickers as humans, in some embodiments, some or all of the steps taken by the picker may be automated. For example, a semi- or fully-autonomous robot may collect items in a retailer location for an order and an autonomous vehicle may deliver an order to a customer from a retailer location.

The retailer computing system 120 is a computing system operated by a retailer that interacts with the online system 140. As used herein, a “retailer” is an entity that operates a “retailer location,” which is a store, warehouse, or other building from which a picker can collect items. The retailer computing system 120 stores and provides item data to the online system 140 and may regularly update the online system 140 with updated item data. For example, the retailer computing system 120 may provide item data indicating which items are available at a retailer location and the quantities of those items. Additionally, the retailer computing system 120 may transmit updated item data to the online system 140 when an item is no longer available at the retailer location. Additionally, the retailer computing system 120 may provide the online system 140 with updated item prices, sales, or availabilities. Additionally, the retailer computing system 120 may receive payment information from the online system 140 for orders serviced by the online system 140. Alternatively, the retailer computing system 120 may provide payment to the online system 140 for some portion of the overall cost of a customer's order (e.g., as a commission).

The customer client device 100, the picker client device 110, the retailer computing system 120, and the online system 140 may communicate with each other via the network 130. The network 130 is a collection of computing devices that communicate via wired or wireless connections. The network 130 may include one or more local area networks (LANs) or one or more wide area networks (WANs). The network 130, as referred to herein, is an inclusive term that may refer to any or all standard layers used to describe a physical or virtual network, such as the physical layer, the data link layer, the network layer, the transport layer, the session layer, the presentation layer, and the application layer. The network 130 may include physical media for communicating data from one computing device to another computing device, such as MPLS lines, fiber optic cables, cellular connections (e.g., 3G, 4G, or 5G spectra), or satellites. The network 130 also may use networking protocols, such as TCP/IP, HTTP, SSH, SMS, or FTP, to transmit data between computing devices. In some embodiments, the network 130 may include Bluetooth or near-field communication (NFC) technologies or protocols for local communications between computing devices. The network 130 may transmit encrypted or unencrypted data.

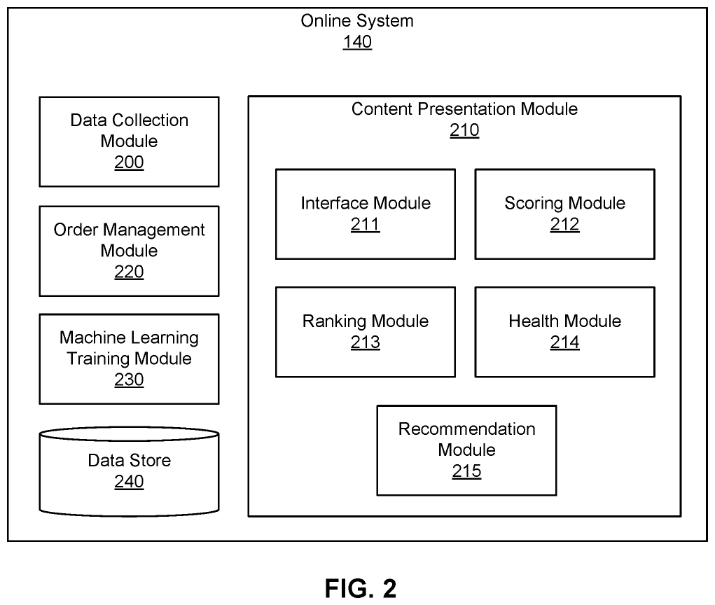

The online system 140 may be an online concierge system by which customers can order items to be provided to them by a picker from a retailer. The online system 140 receives orders from a customer client device 100 through the network 130. The online system 140 selects a picker to service the customer's order and transmits the order to a picker client device 110 associated with the picker. The picker collects the ordered items from a retailer location and delivers the ordered items to the customer. The online system 140 may charge a customer for the order and provide portions of the payment from the customer to the picker and the retailer. As an example, the online system 140 may allow a customer to order groceries from a grocery store retailer. The customer's order may specify which groceries they want delivered from the grocery store and the quantities of each of the groceries. The customer client device 100 transmits the customer's order to the online system 140 and the online system 140 selects a picker to travel to the grocery store retailer location to collect the groceries ordered by the customer. Once the picker has collected the groceries ordered by the customer, the picker delivers the groceries to a location transmitted to the picker client device 110 by the online system 140. The online system 140 is described in further detail below with regards to FIG. 2.

FIG. 2 illustrates an example system architecture for an online system 140, such as an online concierge system, in accordance with some embodiments. The system architecture illustrated in FIG. 2 includes a data collection module 200, a content presentation module 210, an order management module 220, a machine learning training module 230, and a data store 240. Alternative embodiments may include more, fewer, or different components from those illustrated in FIG. 2, and the functionality of each component may be divided between the components differently from the description below. Additionally, each component may perform their respective functionalities in response to a request from a human, or automatically without human intervention.

The data collection module 200 collects data used by the online system 140 and stores the data in the data store 240. The data collection module 200 may only collect data describing a user if the user has previously explicitly consented to the online system 140 collecting data describing the user. Additionally, the data collection module 200 may encrypt all data, including sensitive or personal data, describing users.

The data collection module 200 collects customer data, which is information or data that describe characteristics or other types of information associated with a customer. Customer data may include a customer's name, address, shopping preferences, favorite items, favorite retailers, stored payment instruments, or dietary preferences/restrictions (e.g., vegetarian, gluten-free, peanut allergy, etc.). The customer data also may include default settings established by a customer, such as a default retailer/retailer location, payment instrument, delivery location, or delivery timeframe. Customer data also may include historical interaction information, such as historical order/purchase information associated with a customer. For example, customer data may include information describing previous orders placed by a customer with the online system 140 or information describing previous purchases made by the customer at retailer locations. As an additional example, customer data may include information describing previous interactions by a customer with items, recipes, or other objects presented by the online system 140. In the above example, the customer data may include information describing the objects (e.g., item or recipe types, item prices, recipe ratings, etc.), the types of interactions (e.g., adding items to a shopping list, searching for items or recipes, saving recipes, etc.), and the times of the interactions (e.g., a timestamp associated with each interaction).

Customer data further may include health data associated with a customer. Health data may include various health-related information or metrics, such as a customer's age, gender, body weight, body mass index (BMI), blood pressure, glucose level, iron level, food log, health conditions (e.g., anemia, diabetes, high cholesterol, etc.), health goals (e.g., to increase bone density by a certain amount or to achieve an ideal bone density), or any other suitable types of information. In embodiments in which the health data includes one or more health conditions for a customer, each health condition may be provided by the customer or confirmed by the customer (e.g., once a class associated with the customer describing the health condition has been received from the multiclass classification model, as described below). Health data also may include various types of information determined by the health module 214, as further described below. The data collection module 200 may collect the customer data from sensors on the customer client device 100, from the health module 214, or based on a customer's interactions with the online system 140.

The data collection module 200 also collects item data, which is information or data that identifies and describes items that are available at a retailer location. The item data may include item identifiers for items that are available and may include quantities of items associated with each item identifier. Additionally, item data may also include attributes of items such as the sizes, colors, weights, stock keeping units (SKUs), serial numbers, prices, item categories, brands, sales, discounts, qualities (e.g., freshness, ripeness, etc.), ingredients, materials, manufacturing locations, versions/varieties (e.g., low sodium, gluten-free, etc.), nutritional information, or any other suitable attributes of the items. The item data may further include purchasing rules associated with each item, if they exist. For example, age-restricted items such as alcohol and tobacco are flagged accordingly in the item data. Item data may also include information that is useful for predicting the availability of items at retailer locations. For example, for each item-retailer combination (a particular item at a particular retailer location), the item data may include a time that the item was last found, a time that the item was last not found (a picker looked for the item but could not find it), the rate at which the item is found, or the popularity of the item. The data collection module 200 may collect item data from a retailer computing system 120, a picker client device 110, or a customer client device 100.

An item category is a set of items that are a similar type of item. Items in an item category may be considered to be equivalent to each other or may be replacements for each other in an order. For example, different brands of sourdough bread may be different items, but these items may be in a “sourdough bread” item category. In some embodiments, item categories may be broader in that the same item category may include item types that are related to a common theme, found in the same department, etc. For example, items such as soy sauce, ramen, and miso soup may be included in an “Asian foods” item category. Furthermore, in various embodiments, an item may be included in multiple categories. For example, fresh strawberries may be included in a “strawberries” item category, a “fruit” item category, a “produce” item category, “a fresh strawberries” item category, a “fresh fruit” item category, as well as a “fresh produce” item category. The item categories may be human-generated and human-populated with items. The item categories also may be generated automatically by the online system 140 (e.g., using a clustering algorithm).

The data collection module 200 also collects recipe data, which is information or data describing characteristics of recipes obtained by the online system 140. A recipe may include one or more items, a quantity of each item, and information describing how to combine the items in the recipe. Additionally, recipe data may also include one or more attributes describing recipes, such as recipe categories, ingredients, preparation time, complexity, serving size, nutritional information (e.g., calories, sodium, fat, or sugar content, or other information included in a nutrient profile), ratings, or any other suitable types of information. Attributes of a recipe may be included in the recipe by a source from which the recipe was received or may be determined by the data collection module 200 from ingredients included in the recipe or other information included in the recipe. A recipe category may correspond to a cuisine associated with the recipe (e.g., Thai, Mediterranean, etc.), a meal or food associated with the recipe (e.g., dessert, Thanksgiving, etc.), a diet associated with the recipe (e.g., low-fat, keto, vegan, dairy-free, etc.), a spice level associated with the recipe (e.g., spicy or mild), one or more ingredients featured in the recipe (e.g., rice for risotto), etc. Similar to item categories, recipe categories may be human-generated and human-populated with recipes and also may be generated automatically by the online system 140 (e.g., using a clustering algorithm). The data collection module 200 may collect recipes from users (e.g., customer client devices 100), third-party systems (e.g., websites or applications), or any other suitable source, and stored in the data store 240.

The data collection module 200 also collects picker data, which is information or data describing characteristics of pickers. For example, the picker data for a picker may include the picker's name, the picker's location, how often the picker has serviced orders for the online system 140, a customer rating for the picker, the retailers from which the picker has collected items, or the picker's previous shopping history. Additionally, the picker data may include preferences expressed by the picker, such as their preferred retailers for collecting items, how far they are willing to travel to deliver items to a customer, how many items they are willing to collect at a time, timeframes within which the picker is willing to service orders, or payment information by which the picker is to be paid for servicing orders (e.g., a bank account). The data collection module 200 collects picker data from sensors of the picker client device 110 or from the picker's interactions with the online system 140.

Additionally, the data collection module 200 collects order data, which is information or data describing characteristics of an order. For example, order data may include item data for items that are included in the order, a delivery location for the order, a customer associated with the order, a retailer location from which the customer wants the ordered items collected, or a timeframe within which the customer wants the order delivered. Order data may further include information describing how the order was serviced, such as which picker serviced the order, when the order was delivered, or a rating that the customer gave the delivery of the order.

The content presentation module 210 selects content for presentation to a customer. For example, the content presentation module 210 selects which items to present to a customer while the customer is placing an order. Components of the content presentation module 210 include: an interface module 211, a scoring module 212, a ranking module 213, a health module 214, and a recommendation module 215, which are further described below.

The interface module 211 generates and transmits an ordering interface for a customer to order items. The interface module 211 populates the ordering interface with items that the customer may select for adding to their order. In some embodiments, the interface module 211 presents a catalog of all items that are available to the customer, which the customer can browse to select items to order. Other components of the content presentation module 210 may identify items that the customer is most likely to order and the interface module 211 may then present those items to the customer. For example, the scoring module 212 may score items and the ranking module 213 may rank the items based on their scores. In this example, the recommendation module 215 may select items with scores that exceed some threshold (e.g., the top n items or the p percentile of items) and the interface module 211 then displays the items. The interface module 211 also may generate and transmit an interface including other types of information, as further described below.

The scoring module 212 may use an item selection model to score items for presentation to a customer. An item selection model is a machine learning model that is trained to score items for a customer based on item data for the items and customer data for the customer. For example, the item selection model may be trained to determine a likelihood that the customer will order the item. In some embodiments, the item selection model uses item embeddings describing items and customer embeddings describing customers to score items. These item embeddings and customer embeddings may be generated by separate machine learning models and may be stored in the data store 240.

In some embodiments, the scoring module 212 scores items based on a search query received from the customer client device 100. A search query is text for a word or set of words that indicate items of interest to the customer. The scoring module 212 scores items based on a relatedness of the items to the search query. For example, the scoring module 212 may apply natural language processing (NLP) techniques to the text in the search query to generate a search query representation (e.g., an embedding) that represents characteristics of the search query. The scoring module 212 may use the search query representation to score candidate items for presentation to a customer (e.g., by comparing a search query embedding to an item embedding).

In some embodiments, the scoring module 212 scores items based on a predicted availability of an item. The scoring module 212 may use an availability model to predict the availability of an item. An availability model is a machine learning model that is trained to predict the availability of an item at a retailer location. For example, the availability model may be trained to predict a likelihood that an item is available at a retailer location or may predict an estimated number of items that are available at a retailer location. The scoring module 212 may weight the score for an item based on the predicted availability of the item. Alternatively, items may be filtered out from presentation to a customer based on whether the predicted availability of the item exceeds a threshold.

The health module 214 may interact with the data store 240. The health module 214 may do so by retrieving various types of data from the data store 240, such as health data associated with a customer, historical interaction data associated with a customer, item data, recipe data, etc. The health module 214 also may communicate various types of information to the data collection module 200, which may then store the information in the data store 240. Examples of such types of information include: a frequency distribution of dietary attributes of objects with which a customer previously interacted and a set of health-related temporal features associated with a customer, which may be determined by the health module 214, as further described below. This information may be stored in the data store 240 in association with information identifying a customer associated with the information, one or more times associated with the information (e.g., a time period for which a frequency distribution or a set of health-related temporal features was determined), or any other suitable types of information.

As described above, the health module 214 may determine a frequency distribution of a set of dietary attributes of one or more items, recipes, or other objects with which a customer previously interacted. Examples of dietary attributes include: an amount of an ingredient, nutritional information (e.g., included in a nutrient profile), or any other suitable dietary attributes. The health module 214 may determine the frequency distribution based on various types of data, such as customer data describing objects with which the customer previously interacted and data describing the dietary attributes of each object. For example, based on item data and historical interaction data stored in the data store 240, the health module 214 may determine a proportion or percentage of fat for each item included in one or more previous orders placed by a customer. In the above example, based on recipe data and the historical interaction data stored in the data store 240, the health module 214 also may determine a proportion or percentage of sodium for each ingredient included in one or more recipes previously saved by the customer or which the customer indicated they previously made. In some embodiments, the health module 214 determines the frequency distribution of dietary attributes of objects with which the customer recently interacted (e.g., within the past two weeks).

As also described above, the health module 214 also may determine a set of health-related temporal features associated with a customer. Examples of such health-related temporal features include: changes in health-related information or metrics, such as changes in the customer's weight, BMI, blood pressure, glucose level, iron level, etc. The health module 214 may determine the set of health-related temporal features based on health data associated with the customer (e.g., stored in the data store 240). For example, based on health data associated with a customer stored in the data store 240, the health module 214 may determine the customer's blood pressure or glucose level as a function of frequency by transforming a function of time describing fluctuations in the pressure or level using a transform (e.g., Fourier transform). In some embodiments, the health module 214 determines the set of health-related temporal features recently associated with the customer (e.g., within the past two weeks).

The health module 214 also classifies a customer into a set of classes indicating whether the customer has each of a set of health conditions using a multiclass classification model. The multiclass classification model may be a machine learning model trained to classify the customer into the set of classes. In some embodiments, the multiclass classification model includes multiple machine learning models, in which each machine learning model is trained to classify the customer into one of two classes indicating whether the customer has a particular health condition. In such embodiments, the outputs of the models are combined to generate a multiclass output. For example, the multiclass classification model may include multiple binary classifiers, in which each binary classifier generates an output corresponding to a class indicating whether a customer is likely to have a particular health condition. In the above example, the outputs of the classifiers may be combined to generate a multiclass output indicating whether the customer is likely to have each of multiple health conditions. In some embodiments, the multiclass classification model is a large language model (LLM), which is a trained deep-learning model that generates a textual output based on a prompt.

To use the multiclass classification model, the health module 214 may access the model (e.g., from the data store 240) and apply the model to classify whether a customer has each of a set of health conditions. The multiclass classification model may classify whether the customer has each of the set of health conditions based on a set of historical interaction data for the customer, a set of health data associated with the customer, or any other suitable types of information. In some embodiments, the health module 214 applies the multiclass classification model to a set of inputs, which may include a frequency distribution of a set of dietary attributes of one or more items, recipes, or other objects with which the customer previously interacted, a set of health-related temporal features associated with the customer, etc. For example, the set of inputs may include a proportion or percentage of fat, sodium, and sugar for each item included in one or more previous orders placed by a customer or one or more recipes previously saved by the customer. The health module 214 may then receive an output including a set of classes, in which each class indicates whether the customer has a health condition (e.g., high cholesterol, high blood pressure, diabetes, etc.).

In embodiments in which the multiclass classification model is an LLM, the health module 214 uses the model by generating a prompt that it provides to the model. The prompt may include a set of customer data for a customer (e.g., historical interaction data, health data, etc. provided via Llama Index or any other suitable data framework) and a request for a set of health conditions the customer is likely to have based on the customer data for the customer. The health module 214 then extracts the set of health conditions from a textual output received from the multiclass classification model by applying NLP techniques to the textual output or by using any other suitable technique or combination of techniques. In some embodiments, the multiclass classification model is trained by the machine learning training module 230, as further described below.

In various embodiments, once an output is received from the multiclass classification model indicating a customer has one or more particular health conditions, the interface module 211 generates and transmits an interface describing the health condition(s) for display to a customer client device 100 associated with the customer. The customer may then confirm whether they have the health condition(s) by interacting with the interface. For example, the interface may include a list of symptoms associated with each health condition or the name of each health condition and a checkbox next to each symptom or name that allows a customer to confirm whether they are experiencing each symptom or whether they have each health condition.

The health module 214 also may compute one or more health trend scores for a customer based on various types of customer data for the customer. A health trend score may be computed based on a health goal associated with a customer and the progress of the customer towards achieving the health goal, which may be described in a table, graph, etc. For example, health trend scores may be computed based on health goals associated with a customer, (e.g., lowering their LDL cholesterol level to 100 mg/dL, lowering their blood pressure to 120/80 mm Hg, etc.) and the progress of the customer towards achieving the health goals, such that each score is proportional to the customer's progress towards achieving a corresponding goal. A health trend score also may be computed based on changes in a customer's behavior. For example, a health trend score for a customer may be computed based on changes in the amount of saturated fat included in items ordered by the customer or recipes saved by the customer over a period of time, such that the score increases as the amount of saturated fat decreases. The health module 214 may update a health trend score for a customer as more customer data for the customer is received. For example, suppose that the health module 214 computes the health trend score for a customer based on a weight goal associated with the customer and the progress of the customer towards achieving the weight goal. In this example, the health module 214 may update the health trend score for the customer as an additional set of health data associated with the customer is received from a customer client device 100 associated with the customer (e.g., automatically from a smartwatch or a smart scale or when it is manually entered into a laptop or a tablet by the customer). Once the health module 214 computes or updates a health trend score for a customer, the interface module 211 may generate and send an interface including the health trend score to a customer client device 100 associated with the customer.

The recommendation module 215 may generate a prompt to an LLM that generates a textual output based on the prompt. The prompt may include a set of classes into which a customer was classified by the multiclass classification model and a request for a set of objects for the customer. The request included in the prompt may be for a set of objects appropriate for the customer based on whether the customer has each of a set of health conditions indicated by the set of classes. For example, the prompt may include a set of classes associated with a customer, in which the set of classes indicates whether the customer has each of a set of health conditions and a request for a set of items and/or recipes that are appropriate for the customer, in which an appropriateness of an item or a recipe for the customer is based on whether the customer has each health condition.

In some embodiments, the prompt generated by the recommendation module 215 also includes additional types of information, which may be provided via Llama Index or any other suitable data framework. Examples of such types of information include: customer data associated with the customer (e.g., historical interaction data or health conditions associated with the customer), data describing objects with which the customer may interact (e.g., item data or recipe data stored in the data store 240), or any other suitable types of information. For example, suppose that in the above example, the prompt also includes a set of dietary preferences or restrictions associated with the customer (e.g., vegetarian, gluten-free, peanut allergy, etc.) or one or more health conditions included among customer data stored in the data store 240 not described by the set of classes. In this example, the appropriateness of an item or recipe for the customer also may be based on the set of dietary preferences or restrictions associated with the customer (e.g., no items or recipes including meat if the customer is a vegetarian) or the health condition(s). Alternatively, in the above example, if the prompt also includes historical interaction data for the customer, such as information describing several meat items included in previous orders placed by the customer, the appropriateness of an item or recipe for the customer also may be based on the historical interaction data (e.g., items or recipes may include meat). If the prompt includes information describing one or more objects with which the customer may interact, the request included in the prompt may be for one or more alternatives to the object(s) that are more appropriate for the customer based on whether the customer has each of a set of health conditions indicated by the set of classes. For example, suppose that a customer has requested to add a chocolate cake item to their shopping list or has requested to save a recipe for chocolate cake and that a class associated with the customer indicates that the customer has a health condition corresponding to diabetes. In this example, the prompt generated by the recommendation module 215 may include a description of the chocolate cake item or recipe (e.g., nutritional information) and a request for a set of items/recipes corresponding to healthier alternatives based on the customer's health condition.

Once the recommendation module 215 generates a prompt to the LLM, the recommendation module 215 may provide the prompt to the LLM to obtain a textual output. The textual output may describe one or more objects (e.g., items or recipes) or object categories (e.g., item categories or recipe categories) recommended for a customer based on the prompt. For example, suppose that the prompt includes classes associated with a customer indicating that the customer has health conditions corresponding to diabetes and high cholesterol and a request for a set of items appropriate for the customer based on these health conditions. In this example, the textual output obtained from the LLM may describe dietary guidelines the customer should follow, such as emphasizing whole, unprocessed foods, choosing lean sources of protein, incorporating healthy fats, limiting saturated and trans fats, etc. Continuing with this example, the textual output also may describe item categories associated with the guidelines, such as skinless poultry, fish, legumes, and tofu as examples of lean sources of protein, avocados, nuts, seeds, and olive oil as sources of healthy fats, etc. Alternatively, in the above example, if the request is for a set of recipes appropriate for the customer, the textual output obtained from the LLM may describe recipes that are suitable based on the customer's health conditions with general descriptions for how to prepare them (e.g., grill chicken breast in herbs and serve with a side of roasted vegetables, bake salmon and serve with quinoa and steamed vegetables, etc.). In the above examples, if the prompt to the LLM also includes data stored in the data store 240 describing objects with which the customer may interact, such as item data or recipe data, the textual output also may identify specific objects (e.g., a brand, size, etc. of each recommended item or a name, ingredients, etc. of each recommended recipe).

The recommendation module 215 also extracts, from the textual output obtained from the LLM, one or more objects (e.g., items or recipes) for recommendation to a customer. The recommendation module 215 may extract the object(s) by applying NLP techniques to the textual output or by using any other suitable technique or combination of techniques. For example, if the textual output includes information identifying one or more items or recipes recommended for a customer and one or more items or recipes the customer should limit or avoid, the recommendation module 215 may extract only the one or more items or recipes recommended for the customer. In embodiments in which the textual output describes one or more object categories (e.g., item categories or recipe categories) recommended for the customer, when extracting the object(s) for recommendation to the customer, the recommendation module 215 also identifies one or more objects included among the object categories. For example, if the textual output describes fresh fruits and vegetables, the recommendation module 215 may extract one or more items included in a “fresh fruits” item category and a “fresh vegetables” item category based on information describing items included in the item categories stored in the data store 240. In some embodiments, the recommendation module 215 also extracts the object(s) for recommendation to the customer based on a predicted availability associated with each object. For example, the recommendation module 215 may use the availability model described above to predict the availability of one or more items (e.g., items included as ingredients of a recipe) at a retailer location associated with a customer and extract one or more items or recipes from the textual output based on the predicted availability, such that the extracted items or recipes are associated with at least a threshold predicted availability. Once the recommendation module 215 extracts the object(s) for recommendation to the customer, the interface module 211 generates and transmits an interface including the object(s) for recommendation to the customer. For example, the interface module 211 may generate a recommendation for the object(s) and send the recommendation for display to a customer client device 100 associated with the customer.

The order management module 220 manages orders for items from customers. The order management module 220 receives orders from customer client devices 100 and assigns the orders to pickers for service based on picker data. For example, the order management module 220 assigns an order to a picker based on the picker's location and the retailer location from which the ordered items are to be collected. The order management module 220 may also assign an order to a picker based on how many items are in the order, a vehicle operated by the picker, the delivery location, the picker's preferences for how far to travel to deliver an order, the picker's ratings by customers, or how often a picker agrees to service an order.

In some embodiments, the order management module 220 determines when to assign an order to a picker based on a delivery timeframe requested by the customer who placed the order. The order management module 220 computes an estimated amount of time that it would take for a picker to collect the items for an order and deliver the ordered items to the delivery location for the order. The order management module 220 assigns the order to a picker at a time such that, if the picker immediately services the order, the picker is likely to deliver the order at a time within the timeframe. Thus, when the order management module 220 receives an order, the order management module 220 may delay in assigning the order to a picker if the timeframe is far enough in the future.

When the order management module 220 assigns an order to a picker, the order management module 220 transmits the order to the picker client device 110 associated with the picker. The order management module 220 may also transmit navigation instructions from the picker's current location to the retailer location associated with the order. If the order includes items to collect from multiple retailer locations, the order management module 220 identifies the retailer locations to the picker and may also specify a sequence in which the picker should visit the retailer locations.

The order management module 220 may track the location of the picker through the picker client device 110 to determine when the picker arrives at the retailer location. When the picker arrives at the retailer location, the order management module 220 transmits the order to the picker client device 110 for display to the picker. As the picker uses the picker client device 110 to collect items at the retailer location, the order management module 220 receives item identifiers for items that the picker has collected for the order. In some embodiments, the order management module 220 receives images of items from the picker client device 110 and applies computer-vision techniques to the images to identify the items depicted by the images. The order management module 220 may track the progress of the picker as the picker collects items for an order and may transmit progress updates to the customer client device 100 that describe which items have been collected for the customer's order.

In some embodiments, the order management module 220 tracks the location of the picker within the retailer location. The order management module 220 uses sensor data from the picker client device 110 or from sensors in the retailer location to determine the location of the picker in the retailer location. The order management module 220 may transmit instructions to the picker client device 110 to display a map of the retailer location indicating where in the retailer location the picker is located. Additionally, the order management module 220 may instruct the picker client device 110 to display the locations of items for the picker to collect, and may further display navigation instructions for how the picker can travel from their current location to the location of a next item to collect for an order.

The order management module 220 determines when the picker has collected all of the items for an order. For example, the order management module 220 may receive a message from the picker client device 110 indicating that all of the items for an order have been collected. Alternatively, the order management module 220 may receive item identifiers for items collected by the picker and determine when all of the items in an order have been collected. When the order management module 220 determines that the picker has completed an order, the order management module 220 transmits the delivery location for the order to the picker client device 110. The order management module 220 may also transmit navigation instructions to the picker client device 110 that specify how to travel from the retailer location to the delivery location, or to a subsequent retailer location for further item collection. The order management module 220 tracks the location of the picker as the picker travels to the delivery location for an order, and updates the customer with the location of the picker so that the customer can track the progress of their order. In some embodiments, the order management module 220 computes an estimated time of arrival for the picker at the delivery location and provides the estimated time of arrival to the customer.

In some embodiments, the order management module 220 facilitates communication between the customer client device 100 and the picker client device 110. As noted above, a customer may use a customer client device 100 to send a message to the picker client device 110. The order management module 220 receives the message from the customer client device 100 and transmits the message to the picker client device 110 for presentation to the picker. The picker may use the picker client device 110 to send a message to the customer client device 100 in a similar manner.

The order management module 220 coordinates payment by the customer for the order. The order management module 220 uses payment information provided by the customer (e.g., a credit card number or a bank account) to receive payment for the order. In some embodiments, the order management module 220 stores the payment information for use in subsequent orders by the customer. The order management module 220 computes a total cost for the order and charges the customer that cost. The order management module 220 may provide a portion of the total cost to the picker for servicing the order, and another portion of the total cost to the retailer.

The machine learning training module 230 trains machine learning models used by the online system 140. The online system 140 may use machine learning models to perform functionalities described herein. Example machine learning models include regression models, support vector machines, naïve bayes, decision trees, k-nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers.

Each machine learning model includes a set of parameters. A set of parameters for a machine learning model is used by the machine learning model to process an input. For example, a set of parameters for a linear regression model may include weights that are applied to each input variable in the linear combination that comprises the linear regression model. Similarly, the set of parameters for a neural network may include weights and biases that are applied at each neuron in the neural network. The machine learning training module 230 generates the set of parameters for a machine learning model by “training” the machine learning model. Once trained, the machine learning model uses the set of parameters to transform inputs into outputs.

The machine learning training module 230 trains a machine learning model based on a set of training examples. Each training example includes input data to which the machine learning model is applied to generate an output. For example, each training example may include customer data, picker data, item data, or order data. In some cases, the training examples also include a label which represents an expected output of the machine learning model. In these cases, the machine learning model is trained by comparing its output from input data of a training example to the label for the training example.

The machine learning training module 230 may apply an iterative process to train a machine learning model whereby the machine learning training module 230 trains the machine learning model on each of the set of training examples. To train a machine learning model based on a training example, the machine learning training module 230 applies the machine learning model to the input data in the training example to generate an output. The machine learning training module 230 scores the output from the machine learning model using a loss function. A loss function is a function that generates a score for the output of the machine learning model such that the score is higher when the machine learning model performs poorly and lower when the machine learning model performs well. In situations in which the training example includes a label, the loss function is also based on the label for the training example. Some example loss functions include the mean square error function, the mean absolute error, the hinge loss function, and the cross-entropy loss function. The machine learning training module 230 updates the set of parameters for the machine learning model based on the score generated by the loss function. For example, the machine learning training module 230 may apply gradient descent to update the set of parameters.

In embodiments in which the health module 214 accesses a multiclass classification model that is trained to classify a customer into a set of classes indicating whether the customer has each of a set of health conditions, the machine learning training module 230 may train the multiclass classification model. The machine learning training module 230 may train the multiclass classification model via supervised learning based on various types of data received by the data collection module 200 and stored in the data store 240, such as historical interaction data describing objects with which various customers previously interacted, data for the objects (e.g., item data or recipe data), health data for the customers, or any other suitable types of information. As described above, in some embodiments, the multiclass classification model includes multiple machine learning models, in which each machine learning model is trained to classify the customer into one of two classes indicating whether the customer has a particular health condition. In such embodiments, each machine learning model may be trained by the machine learning training module 230. As also described above, in some embodiments, the multiclass classification model is an LLM. In such embodiments, the machine learning training module 230 trains the multiclass classification model via supervised, semi-supervised, self-supervised, unsupervised, or any other suitable learning technique or techniques.

To illustrate an example of how the multiclass classification model may be trained, suppose that the machine learning training module 230 receives a set of training examples. In this example, the set of training examples may include attributes of customers, such as a frequency distribution of a set of dietary attributes of one or more items, recipes, or other objects with which each customer previously interacted and a set of health-related temporal features associated with each customer determined by the health module 214 based on various types of data stored in the data store 240, as described above. In the above example, the machine learning training module 230 also may receive labels which represent expected outputs of the multiclass classification model or each machine learning model included in the multiclass classification model, in which a label indicates an existence of a health condition associated with a customer. Continuing with this example, the machine learning training module 230 may then train the multiclass classification model or each machine learning model included in the multiclass classification model based on the attributes as well as the labels by comparing its output from input data of each training example to the label for the training example.

The data store 240 stores data used by the online system 140. For example, the data store 240 stores customer data, item data, recipe data, order data, and picker data, for use by the online system 140. The data store 240 also stores trained machine learning models trained by the machine learning training module 230. For example, the data store 240 may store the set of parameters for a trained machine learning model on one or more non-transitory, computer-readable media. The data store 240 uses computer-readable media to store data, and may use databases to organize the stored data.

Recommending Items or Recipes Based on Health Conditions Associated with Online System Users

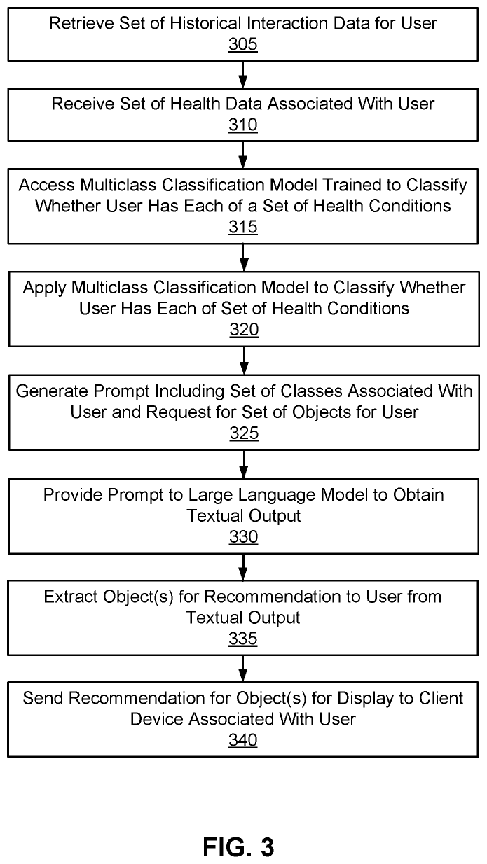

FIG. 3 is a flowchart of a method for recommending items or recipes based on health conditions associated with online system users, in accordance with one or more embodiments. Alternative embodiments may include more, fewer, or different steps from those illustrated in FIG. 3, and the steps may be performed in a different order from that illustrated in FIG. 3. These steps may be performed by an online system (e.g., online system 140), such as an online concierge system. Additionally, each of these steps may be performed automatically by the online system 140 without human intervention.

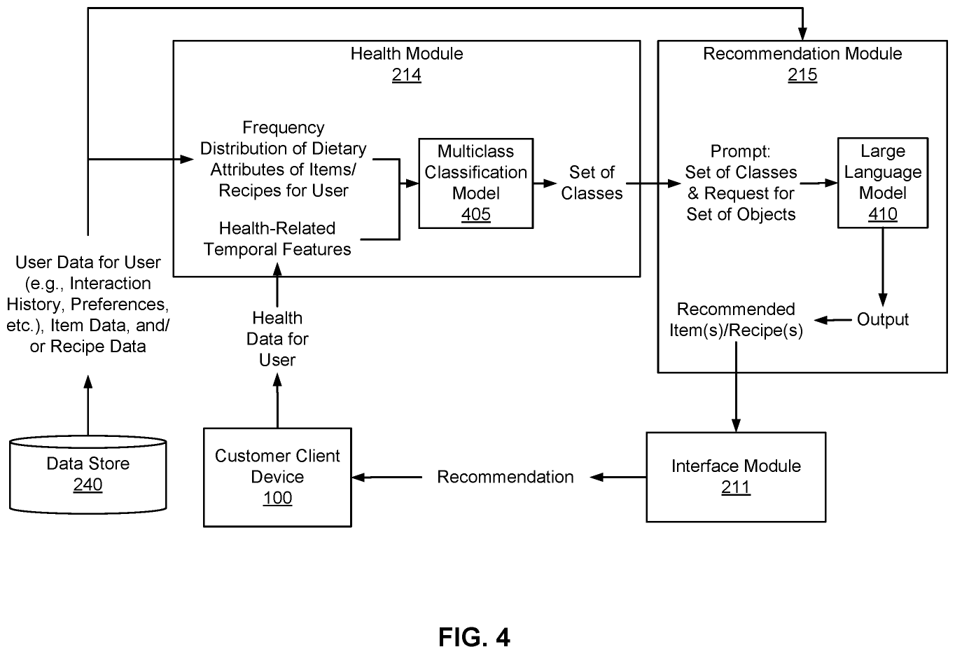

The online system 140 retrieves 305 (e.g., from the data store 240 using the health module 214) a set of historical interaction data for a customer describing a set of objects with which the customer previously interacted (e.g., over the past n days). The set of historical interaction data may include historical order/purchase information associated with the customer. For example, the set of historical interaction data for the customer may describe previous orders placed by the customer with the online system 140 or information describing previous purchases made by the customer at retailer locations. As an additional example, the set of historical interaction data for the customer may describe previous interactions by the customer with items, recipes, or other objects presented by the online system 140. In the above example, the set of historical interaction data for the customer also may describe the objects (e.g., item or recipe types, item prices, recipe ratings, etc.), the types of interactions (e.g., adding items to a shopping list, searching for items or recipes, saving recipes, etc.), and the times of the interactions (e.g., a timestamp associated with each interaction). In some embodiments, the online system 140 also retrieves (step 305) additional types of information in association with the set of historical interaction data for the customer. FIG. 4 is an interaction diagram of a method for recommending items or recipes based on health conditions associated with online system users, in accordance with one or more embodiments. As shown in FIG. 4, the online system 140 may retrieve 305 the set of historical interaction data for the customer in association with other customer data, such as a set of preferences (e.g., dietary preferences) associated with the customer, item data, and recipe data.

Referring back to FIG. 3, the online system 140 receives 310 (e.g., via the data collection module 200) a set of health data associated with the customer. The set of health data associated with the customer may include various health-related information or metrics, such as the customer's age, gender, body weight, body mass index (BMI), blood pressure, glucose level, iron level, food log, health conditions (e.g., anemia, diabetes, high cholesterol, etc.), health goals (e.g., to increase bone density by a certain amount or to achieve an ideal bone density), or any other suitable types of information. In embodiments in which the set of health data includes one or more health conditions for the customer, each health condition may be provided by the customer or confirmed by the customer (e.g., once a class associated with the customer describing the health condition has been received from the multiclass classification model, as described below). The set of health data also may include various types of information determined by the online system 140 (e.g., using the health module 214), such as information describing a frequency distribution of dietary attributes of objects with which the customer previously interacted, a set of health-related temporal features associated with the customer, etc., as further described below. As shown in FIG. 4, the online system 140 may receive 310 the set of health data associated with the customer from a customer client device 100 associated with the customer. In this example, the customer client device 100 may be a personal or mobile computing device, such as a smartphone, a tablet, a laptop computer, or a desktop computer. Alternatively, In the above example, the customer client device 100 may be a wearable device (e.g., a smartwatch, a fitness tracker, etc.) or another device (e.g., a smart scale) that may interact with the online system 140 either directly or indirectly (e.g., via a Bluetooth connection with another customer client device 100 or a third-party system, such as a fitness tracking application, that communicates with the online system 140).

In some embodiments, the online system 140 determines (e.g., using the health module 214) a frequency distribution of a set of dietary attributes of one or more items, recipes, or other objects with which the customer previously interacted. Examples of dietary attributes include: an amount of an ingredient, nutritional information (e.g., included in a nutrient profile), or any other suitable dietary attributes. The online system 140 may determine the frequency distribution based on various types of data, such as the set of historical interaction data for the customer describing the set of objects with which the customer previously interacted and data describing the dietary attributes of each object. For example, based on item data and the retrieved 305 set of historical interaction data for the customer, the online system 140 may determine a proportion or percentage of fat for each item included in one or more previous orders placed by the customer. In the above example, based on recipe data and the set of historical interaction data, the online system 140 also may determine a proportion or percentage of sodium for each ingredient included in one or more recipes previously saved by the customer or which the customer indicated they previously made. In some embodiments, the online system 140 determines the frequency distribution of dietary attributes of objects with which the customer recently interacted (e.g., within the past two weeks). The online system 140 may store information describing the frequency distribution of dietary attributes of objects with which the customer previously interacted (e.g., in the data store 240) in association with information identifying the customer, a time associated with the frequency distribution (e.g., a time period for which it was determined), or any other suitable types of information.

In various embodiments, the online system 140 also determines (e.g., using the health module 214) a set of health-related temporal features associated with the customer. Examples of such health-related temporal features include: changes in health-related information or metrics, such as changes in the customer's weight, BMI, blood pressure, glucose level, iron level, etc. The online system 140 may determine the set of health-related temporal features based on the received 310 set of health data associated with the customer. For example, based on the set of health data associated with the customer, the online system 140 may determine the customer's blood pressure or glucose level as a function of frequency by transforming a function of time describing fluctuations in the pressure or level using a transform (e.g., Fourier transform). In some embodiments, the online system 140 determines the set of health-related temporal features recently associated with the customer (e.g., within the past two weeks). The online system 140 may store information describing the set of health-related temporal features associated with the customer (e.g., in the data store 240) in association with information identifying the customer, at time associated with the set of health-related temporal features (e.g., a time period for which it was determined), or any other suitable types of information.

Referring again to FIG. 3, the online system 140 then classifies (e.g., using the health module 214) the customer into a set of classes indicating whether the customer has each of a set of health conditions using a multiclass classification model. The multiclass classification model may be a machine learning model trained to classify the customer into the set of classes. In some embodiments, the multiclass classification model includes multiple machine learning models, in which each machine learning model is trained to classify the customer into one of two classes indicating whether the customer has a particular health condition. In such embodiments, the outputs of the models are combined to generate a multiclass output. For example, the multiclass classification model may include multiple binary classifiers, in which each binary classifier generates an output corresponding to a class indicating whether the customer is likely to have a particular health condition. In the above example, the outputs of the classifiers may be combined to generate a multiclass output indicating whether the customer is likely to have each of multiple health conditions. In some embodiments, the multiclass classification model is a large language model (LLM), which is a trained deep-learning model that generates a textual output based on a prompt.

To use the multiclass classification model, the online system 140 may access 315 (e.g., using the health module 214) the model (e.g., from the data store 240) and apply 320 (e.g., using the health module 214) the model to classify whether the customer has each of the set of health conditions. The multiclass classification model may classify whether the customer has each of the set of health conditions based on the set of historical interaction data for the customer, the set of health data associated with the customer, or any other suitable types of information. As shown in the example of FIG. 4, the online system 140 also may apply 320 the multiclass classification model 405 to a set of inputs, which may include the frequency distribution of a set of dietary attributes of one or more items, recipes, or other objects with which the customer previously interacted (described above) and the set of health-related temporal features associated with the customer (also described above). The online system 140 may then receive an output including a set of classes, in which each class indicates whether the customer has a health condition (e.g., high cholesterol, high blood pressure, diabetes, etc.).

In embodiments in which the multiclass classification model 405 is an LLM, the online system 140 uses the model 405 by generating (e.g., using the health module 214) a prompt that it provides to the model 405. The prompt may include a set of customer data for the customer (e.g., the set of historical interaction data, the set of health data, etc. provided via Llama Index or any other suitable data framework) and a request for the set of health conditions the customer is likely to have based on the customer data for the customer. The online system 140 then extracts (e.g., using the health module 214) the set of health conditions from a textual output received from the multiclass classification model 405 by applying NLP techniques to the textual output or by using any other suitable technique or combination of techniques.

In various embodiments, once an output is received from the multiclass classification model 405 indicating the customer has one or more particular health conditions, the online system 140 generates and transmits (e.g., using the interface module 211) an interface describing the health condition(s) for display to the customer client device 100 associated with the customer. The customer may then confirm whether they have the health condition(s) by interacting with the interface. For example, the interface may include a list of symptoms associated with each health condition or the name of each health condition and a checkbox next to each symptom or name that allows the customer to confirm whether they are experiencing each symptom or whether they have each health condition.

In some embodiments, the multiclass classification model 405 is trained by the online system 140 (e.g., by the machine learning training module 230). The online system 140 may train the multiclass classification model 405 via supervised learning based on various types of data received by the online system 140 (e.g., via the data collection module 200) and stored (e.g., in the data store 240). Examples of such types of data include: historical interaction data describing objects with which various customers previously interacted, data for the objects (e.g., item data or recipe data), health data for the customers, or any other suitable types of information. As described above, in some embodiments, the multiclass classification model 405 includes multiple machine learning models, in which each machine learning model is trained to classify the customer into one of two classes indicating whether the customer has a particular health condition. In such embodiments, each machine learning model may be trained by the online system 140. In embodiments in which the multiclass classification model 405 is an LLM, the online system 140 trains the multiclass classification model 405 via supervised, semi-supervised, self-supervised, unsupervised, or any other suitable learning technique or techniques.

To illustrate an example of how the multiclass classification model 405 may be trained, suppose that the online system 140 receives a set of training examples. In this example, the set of training examples may include attributes of customers, such as a frequency distribution of a set of dietary attributes of one or more items, recipes, or other objects with which each customer previously interacted and a set of health-related temporal features associated with each customer determined by the online system 140 based on various types of data (e.g., stored in the data store 240), as described above. In the above example, the online system 140 also may receive labels which represent expected outputs of the multiclass classification model 405 or each machine learning model included in the multiclass classification model 405, in which a label indicates an existence of a health condition associated with a customer. Continuing with this example, the online system 140 may then train the multiclass classification model 405 or each machine learning model included in the multiclass classification model 405 based on the attributes as well as the labels by comparing its output from input data of each training example to the label for the training example.

Referring back to FIG. 3, the online system 140 then generates 325 (e.g., using the recommendation module 215) a prompt to an LLM that generates a textual output based on the prompt. The prompt may include the set of classes into which the customer was classified by the multiclass classification model 405 and a request for a set of objects for the customer. The request included in the prompt may be for a set of objects appropriate for the customer based on whether the customer has each of the set of health conditions indicated by the set of classes. For example, as shown in FIG. 4, the prompt to the LLM 410 may include the set of classes associated with the customer and a request for a set of items and/or recipes that are appropriate for the customer, in which an appropriateness of an item or a recipe for the customer is based on whether the customer has each health condition.

In some embodiments, the prompt generated 325 by the online system 140 also includes additional types of information, which may be provided via Llama Index or any other suitable data framework. Examples of such types of information include: customer data associated with the customer (e.g., historical interaction data or health conditions associated with the customer), data describing objects with which the customer may interact (e.g., item data or recipe data stored in the data store 240), or any other suitable types of information. For example, suppose that in the above example, the prompt also includes a set of dietary preferences or restrictions associated with the customer (e.g., vegetarian, gluten-free, peanut allergy, etc.) or one or more health conditions included among customer data (e.g., stored in the data store 240) not described by the set of classes. In this example, the appropriateness of an item or recipe for the customer also may be based on the set of dietary preferences or restrictions associated with the customer (e.g., no items or recipes including meat if the customer is a vegetarian) or the health condition(s). Alternatively, in the above example, if the prompt also includes historical interaction data for the customer, such as information describing several meat items included in previous orders placed by the customer, the appropriateness of an item or recipe for the customer also may be based on the historical interaction data (e.g., items or recipes may include meat). If the prompt includes information describing one or more objects with which the customer may interact, the request included in the prompt may be for one or more alternatives to the object(s) that are more appropriate for the customer based on whether the customer has each of the set of health conditions indicated by the set of classes. For example, suppose that the customer has requested to add a chocolate cake item to their shopping list or has requested to save a recipe for chocolate cake and that a class associated with the customer indicates that the customer has a health condition corresponding to diabetes. In this example, the prompt generated 325 by the online system 140 may include a description of the chocolate cake item or recipe (e.g., nutritional information) and a request for a set of items/recipes corresponding to healthier alternatives based on the customer's health condition.

Referring again to FIG. 3, once the online system 140 generates 325 the prompt to the LLM 410, the online system 140 may provide 330 (e.g., using the recommendation module 215) the prompt to the LLM 410 to obtain a textual output. The textual output may describe one or more objects (e.g., items or recipes) or object categories (e.g., item categories or recipe categories) recommended for the customer based on the prompt. For example, as shown in FIG. 4, suppose that the prompt includes classes associated with the customer indicating that the customer has health conditions corresponding to diabetes and high cholesterol and a request for a set of items appropriate for the customer based on these health conditions. In this example, the textual output obtained from the LLM 410 may describe dietary guidelines the customer should follow, such as emphasizing whole, unprocessed foods, choosing lean sources of protein, incorporating healthy fats, limiting saturated and trans fats, etc. Continuing with this example, the textual output also may describe item categories associated with the guidelines, such as skinless poultry, fish, legumes, and tofu as examples of lean sources of protein, avocados, nuts, seeds, and olive oil as sources of healthy fats, etc. Alternatively, in the above example, if the request is for a set of recipes, the textual output obtained from the LLM 410 may describe recipes that are suitable based on the customer's health conditions with general descriptions for how to prepare them (e.g., grill chicken breast in herbs and serve with a side of roasted vegetables, bake salmon and serve with quinoa and steamed vegetables, etc.). In the above examples, if the prompt to the LLM 410 also includes data (e.g., stored in the data store 240) describing objects with which the customer may interact, such as item data or recipe data, the textual output also may identify specific objects (e.g., a brand, size, etc. of each recommended item or a name, ingredients, etc. of each recommended recipe).

Referring back to FIG. 3, the online system 140 extracts 335 (e.g., using the recommendation module 215), from the textual output obtained from the LLM 410, one or more objects (e.g., items or recipes) for recommendation to the customer. The online system 140 may extract 335 the object(s) by applying NLP techniques to the textual output or by using any other suitable technique or combination of techniques. For example, as shown in FIG. 4, if the textual output includes information identifying one or more items or recipes recommended for the customer and one or more items or recipes the customer should limit or avoid, the online system 140 may extract 335 only the one or more items or recipes recommended for the customer. In embodiments in which the textual output describes one or more object categories (e.g., item categories or recipe categories) recommended for the customer, when extracting 335 the object(s) for recommendation to the customer, the online system 140 also identifies one or more objects included among the object categories. For example, if the textual output describes fresh fruits and vegetables, the online system 140 may extract 335 one or more items included in a “fresh fruits” item category and a “fresh vegetables” item category based on information describing items included in the item categories (e.g., stored in the data store 240). In some embodiments, the online system 140 also extracts 335 the object(s) for recommendation to the customer based on a predicted availability associated with each object. For example, the online system 140 may use the availability model described above to predict the availability of one or more items (e.g., items included as ingredients of a recipe) at a retailer location associated with the customer and extract 335 one or more items or recipes from the textual output based on the predicted availability, such that the extracted items or recipes are associated with at least a threshold predicted availability.

Referring once more to FIG. 3, once the online system 140 extracts 335 the object(s) for recommendation to the customer, the online system 140 sends 340 (e.g., using the interface module 211) an interface including the object(s) for recommendation to the customer. For example, as shown in FIG. 4, the online system 140 may generate a recommendation for the object(s) and send 340 the recommendation for display to the customer client device 100 associated with the customer.

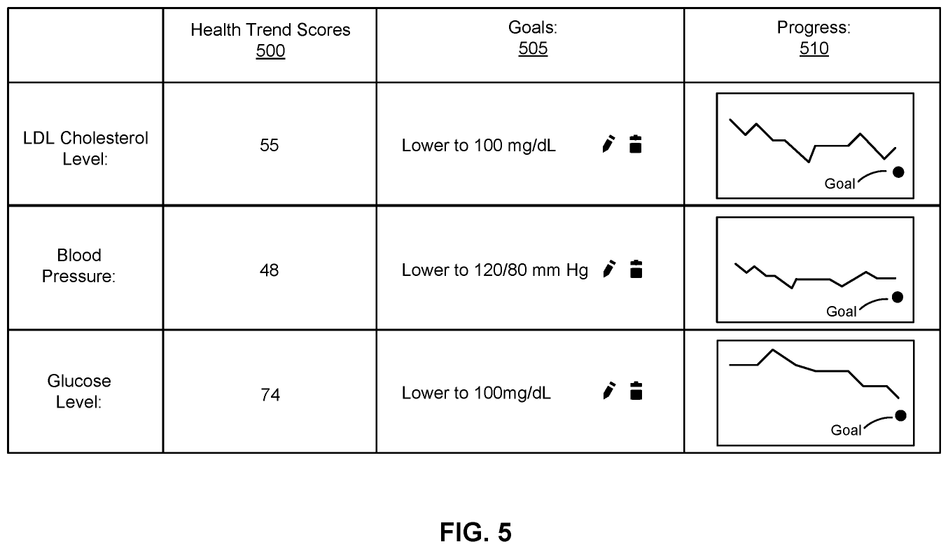

In some embodiments, the online system 140 computes (e.g., using the health module 214) one or more health trend scores for the customer based on various types of customer data for the customer. A health trend score may be computed based on a health goal associated with the customer and the progress of the customer towards achieving the health goal, which may be described in a table, graph, etc. FIG. 5 illustrates an example of health trend scores 500 for a user of an online system 140, in accordance with one or more embodiments. As shown in this example, the health trend scores 500 may be computed based on health goals 505 associated with the customer (e.g., lowering their LDL cholesterol level to 100 mg/dL, lowering their blood pressure to 120/80 mm Hg, etc.) and the progress 510 of the customer towards achieving the health goals 505, such that each score 500 is proportional to the customer's progress 510 towards achieving a corresponding goal 505. A health trend score 500 also may be computed based on changes in the customer's behavior. For example, a health trend score 500 for the customer may be computed based on changes in the amount of saturated fat included in items ordered by the customer or recipes saved by the customer over a period of time, such that the score 500 increases as the amount of saturated fat decreases. The online system 140 may update the health trend score(s) 500 for the customer as more customer data for the customer is received. For example, suppose that the online system 140 computes the health trend score 500 for the customer based on a weight goal 505 associated with the customer and the progress 510 of the customer towards achieving the weight goal 505. In this example, the online system 140 may update the health trend score 500 for the customer as an additional set of health data associated with the customer is received 310 from the customer client device 100 associated with the customer (e.g., automatically from a smartwatch or a smart scale or when it is manually entered into a laptop or a tablet by the customer). Once the online system 140 computes or updates the health trend score 500 for the customer, the online system 140 may generate (e.g., using the interface module 211) and send (e.g., using the interface module 211) an interface including the health trend score 500 to the customer client device 100 associated with the customer.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor comprises one or more processors or processing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine learning models in the performance of their described functionalities. A “machine learning model,” as used herein, comprises one or more machine learning models that perform the described functionality. Machine learning models may be stored on one or more computer-readable media with a set of weights. These weights are parameters used by the machine learning model to transform input data received by the model into output data. The weights may be generated through a training process, whereby the machine learning model is trained based on a set of training examples and labels associated with the training examples. The training process may include: applying the machine learning model to a training example, comparing an output of the machine learning model to the label associated with the training example, and updating weights associated with the machine learning model through a back-propagation process. The weights may be stored on one or more computer-readable media, and are used by a system when applying the machine learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or.” For example, a condition “A or B” is satisfied by any one of the following: A is true (or present) and B is false (or not present), A is false (or not present) and B is true (or present), and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A method, performed at a computer system comprising a processor and a computer-readable medium, comprising:
retrieving, at an online system, a set of historical interaction data for a user of the online system describing a set of objects with which the user previously interacted;
receiving a set of health data associated with the user;
accessing a multiclass classification model trained to classify whether the user has each health condition of a set of health conditions, wherein the multiclass classification model is trained by:
receiving historical interaction data describing a plurality of objects with which a plurality of users of the online system previously interacted,
receiving health data for the plurality of users,
receiving a set of labels for each user of the plurality of users, wherein each label of the set of labels indicates an existence of a health condition associated with a corresponding user, and
training the multiclass classification model based at least in part on the historical interaction data, the health data, and the label for each user of the plurality of users;
applying the multiclass classification model to classify whether the user has each health condition of a set of health conditions based at least in part on the set of historical interaction data for the user and the set of health data associated with the user;
generating a prompt comprising a set of classes associated with the user and a request for a set of objects appropriate for the user, wherein the set of classes indicates whether the user has each health condition of the set of health conditions and an appropriateness of an object for the user is based at least in part on whether the user has each health condition of the set of health conditions;
providing the prompt to a large language model to obtain a textual output;
extracting, from the textual output of the large language model, one or more objects for recommendation to the user, wherein the one or more objects comprise one or more of an item or a recipe; and
sending a recommendation for the one or more objects for display to a client device associated with the user.

2. The method of claim 1, wherein extracting the one or more objects for recommendation to the user comprises:
predicting an availability of each item included among the one or more objects at a retailer location; and
extracting the one or more objects for recommendation to the user based at least in part on the predicted availability of each item included among the one or more objects at the retailer location.

3. The method of claim 1, wherein the prompt further comprises a set of user data for the user, and wherein the set of user data describes one or more of: a set of preferences associated with the user or the set of historical interaction data for the user.

4. The method of claim 3, further comprising:
determining, based at least in part on the set of historical interaction data for the user and information describing each item of a plurality of items, a frequency distribution of a set of dietary attributes of each item included in one or more previous orders placed by the user.

5. The method of claim 4, wherein applying the multiclass classification model to classify whether the user has each health condition of the set of health conditions comprises applying the multiclass classification model to the frequency distribution of the set of dietary attributes of each item included in the one or more previous orders placed by the user.

6. The method of claim 5, wherein determining, the frequency distribution of the set of dietary attributes of each item included in the one or more previous orders placed by the user comprises determining a frequency distribution of one or more of: an amount of an ingredient in each item included in the one or more previous orders placed by the user or nutritional information associated with each item included in the one or more previous orders placed by the user.

7. The method of claim 1, further comprising:
determining, based at least in part on the set of health data associated with the user, a set of health-related temporal features associated with the user.

8. The method of claim 7, wherein applying the multiclass classification model to classify whether the user has each health condition of the set of health conditions comprises applying the multiclass classification model to the set of health-related temporal features associated with the user.

9. The method of claim 1, further comprising:
generating a health trend score for the user based at least in part on the set of health data associated with the user, wherein the set of health data describes a health goal associated with the user and a progress of the user towards achieving the health goal; and
sending the health trend score for the user for display to the client device associated with the user.

10. The method of claim 9, further comprising:
receiving an additional set of health data associated with the user;
updating the health trend score for the user based at least in part on the additional set of health data associated with the user; and
sending the updated health trend score for the user for display to the client device associated with the user.

11. A computer program product comprising a non-transitory computer-readable storage medium having instructions encoded thereon that, when executed by a processor, cause the processor to perform steps comprising:
retrieving, at an online system, a set of historical interaction data for a user of the online system describing a set of objects with which the user previously interacted;
receiving a set of health data associated with the user;
accessing a multiclass classification model trained to classify whether the user has each health condition of a set of health conditions, wherein the multiclass classification model is trained by:
receiving historical interaction data describing a plurality of objects with which a plurality of users of the online system previously interacted,
receiving health data for the plurality of users,
receiving a set of labels for each user of the plurality of users, wherein each label of the set of labels indicates an existence of a health condition associated with a corresponding user, and
training the multiclass classification model based at least in part on the historical interaction data, the health data, and the label for each user of the plurality of users;
applying the multiclass classification model to classify whether the user has each health condition of a set of health conditions based at least in part on the set of historical interaction data for the user and the set of health data associated with the user;
generating a prompt comprising a set of classes associated with the user and a request for a set of objects appropriate for the user, wherein the set of classes indicates whether the user has each health condition of the set of health conditions and an appropriateness of an object for the user is based at least in part on whether the user has each health condition of the set of health conditions;
providing the prompt to a large language model to obtain a textual output;
extracting, from the textual output of the large language model, one or more objects for recommendation to the user, wherein the one or more objects comprise one or more of an item or a recipe; and
sending a recommendation for the one or more objects for display to a client device associated with the user.

12. The computer program product of claim 11, wherein extracting the one or more objects for recommendation to the user comprises:
predicting an availability of each item included among the one or more objects at a retailer location; and
extracting the one or more objects for recommendation to the user based at least in part on the predicted availability of each item included among the one or more objects at the retailer location.

13. The computer program product of claim 11, wherein the prompt further comprises a set of user data for the user, and wherein the set of user data describes one or more of: a set of preferences associated with the user or the set of historical interaction data for the user.

14. The computer program product of claim 13, wherein the non-transitory computer-readable storage medium further has instructions encoded thereon that, when executed by the processor, cause the processor to perform steps comprising:
determining, based at least in part on the set of historical interaction data for the user and information describing each item of a plurality of items, a frequency distribution of a set of dietary attributes of each item included in one or more previous orders placed by the user.

15. The computer program product of claim 14, wherein applying the multiclass classification model to classify whether the user has each health condition of the set of health conditions comprises applying the multiclass classification model to the frequency distribution of the set of dietary attributes of each item included in the one or more previous orders placed by the user.

16. The computer program product of claim 15, wherein determining, the frequency distribution of the set of dietary attributes of each item included in the one or more previous orders placed by the user comprises determining a frequency distribution of one or more of: an amount of an ingredient in each item included in the one or more previous orders placed by the user or nutritional information associated with each item included in the one or more previous orders placed by the user.

17. The computer program product of claim 11, wherein the non-transitory computer-readable storage medium further has instructions encoded thereon that, when executed by the processor, cause the processor to perform steps comprising:
determining, based at least in part on the set of health data associated with the user, a set of health-related temporal features associated with the user.

18. The computer program product of claim 17, wherein applying the multiclass classification model to classify whether the user has each health condition of the set of health conditions comprises applying the multiclass classification model to the set of health-related temporal features associated with the user.

19. The computer program product of claim 11, wherein the non-transitory computer-readable storage medium further has instructions encoded thereon that, when executed by the processor, cause the processor to perform steps comprising:
generating a health trend score for the user based at least in part on the set of health data associated with the user, wherein the set of health data describes a health goal associated with the user and a progress of the user towards achieving the health goal;
sending the health trend score for the user for display to the client device associated with the user;
receiving an additional set of health data associated with the user;
updating the health trend score for the user based at least in part on the additional set of health data associated with the user; and
sending the updated health trend score for the user for display to the client device associated with the user.

20. A computer system comprising:
a processor; and
a non-transitory computer-readable storage medium storing instructions that, when executed by the processor, perform actions comprising:
retrieving, at an online system, a set of historical interaction data for a user of the online system describing a set of objects with which the user previously interacted;
receiving a set of health data associated with the user;
accessing a multiclass classification model trained to classify whether the user has each health condition of a set of health conditions, wherein the multiclass classification model is trained by:
receiving historical interaction data describing a plurality of objects with which a plurality of users of the online system previously interacted,
receiving health data for the plurality of users,
receiving a set of labels for each user of the plurality of users, wherein each label of the set of labels indicates an existence of a health condition associated with a corresponding user, and
training the multiclass classification model based at least in part on the historical interaction data, the health data, and the label for each user of the plurality of users;
applying the multiclass classification model to classify whether the user has each health condition of a set of health conditions based at least in part on the set of historical interaction data for the user and the set of health data associated with the user;
generating a prompt comprising a set of classes associated with the user and a request for a set of objects appropriate for the user, wherein the set of classes indicates whether the user has each health condition of the set of health conditions and an appropriateness of an object for the user is based at least in part on whether the user has each health condition of the set of health conditions;
providing the prompt to a large language model to obtain a textual output;
extracting, from the textual output of the large language model, one or more objects for recommendation to the user, wherein the one or more objects comprise one or more of an item or a recipe; and
sending a recommendation for the one or more objects for display to a client device associated with the user.
